(19) United States
(12) Patent Application Publication
(10) Pub. No.: US 2011/0264084 A1
(43) Pub. Date: Oct. 27, 2011

(54) VACUUM INSULATED COOLING PROBE WITH HEAT EXCHANGER

(75) Inventor: Aarne H. Reid, Jupiter, FL (US)

(73) Assignee: Concept Group, Inc., West Berlin, NJ (US)

(21) Appl. No.: 12/766,397

(22) Filed: Apr. 23, 2010

(51) Int. Cl. A61B 18/02

(52) U.S. Cl. 606/23

(57) ABSTRACT

An insulated cooling probe including a probe sleeve assembly having an annular insulating jacket shaped in the vicinity of an evacuation vent to achieve a deeper vacuum within the insulating space than is applied to the vent, and a coolant inlet passageway and a coolant exit passageway bounded by a coolant passageway wall disposed within the insulating jacket, the probe also including a cooling tip extending outwardly from the sleeve assembly at one end of the probe and including a cooling region into which coolant enters from the coolant inlet passageway and from which coolant exits into the coolant exit passageway. The coolant expands across an orifice when upon exiting the coolant inlet passageway and entering the cooling tip. The coolant flowing in the coolant inlet passageway is pre-cooled in a heat transfer region of the probe by transferring heat to coolant flowing in the coolant exit passageway.

### BACKGROUND

A cooling probe is disclosed having an insulating sheath that is evacuated to a high vacuum and a high heat exchanger for enhancing the level of cooling achieved in the probe tip. It is well known that vacuum provides an excellent thermal insulator. Vacuum-sealed spaces have been incorporated in a wide variety of structures including cryogenic devices, such as medical probes, and high temperature devices, such as heat exchangers. Structures including vacuum-sealed spaces, and methods for achieving high levels of vacuum in such spaces, are disclosed in commonly owned U.S. Pat. Nos. 7,374,063 (issued May 20, 2008) and 7,681,299 (issued Mar. 23, 2010).

Cryogenic probes for cooling applications, particular in medical devices, are also known. Numerous cryogenic probes use the Joule-Thompson effect to achieve cooling, while others attempt to use cryogenic liquid within the probe. A challenge with all of these probes has been maintaining the cryogenic fluid (liquid or gas) sufficiently cool as it is transported from a remote console to a handpiece and ultimately to a cooling tip for use in chilling a target surface or tissue, so as to efficiently and effectively deliver cooling to the target surface or tissue without excessive heat loss. In many earlier attempts in which the probe was not adequately insulated along its exterior surface, a supply of cryogenic fluid is circulated in a central tube within the probe from the console to the cooling tip, and the return (warmer) cryogenic fluid or expanded gas is returned via an outer annulus. The return fluid in the outer annulus serves to partially protect or insulate the supply fluid from external heating, but typically takes on significant heat in the process, increasing the amount of energy that must be extracted at the console so that the return fluid can be recycled as supply fluid.

Another challenge with such probes is achieving high cooling efficiencies in view of the flow limitations inherent in their designs. In a cooling probe that relies, at least in part, on expansion of the supply coolant to achieve low temperatures, a pressure differential must exist between the supply coolant and the return coolant. In general terms, the larger the pressure ratio between the supply and return coolants, the larger the cooling effect that can be achieved. In addition, to further enhance the cooling capabilities of a cooling probe, the colder expanded coolant can be used, after passing through the cooling tip, to decrease the temperature of the incoming supply coolant. Existing probes often utilize complex heat exchangers and precoolers, located in the cooling tip itself or in a handle distal from the cooling tip, which cause significant frictional flow losses in the return coolant (translating into back-pressure in the cooling tip).

Additionally, because in many conventional cooling probes the supply coolant flows in the central tube and the return coolant flows in the annular space between the central tube and a jacket, for a fixed diameter jacket (i.e., a probe having a fixed external diameter) there is an inverse relationship between the annular cross-sectional area provided for the return coolant to flow and the surface area of the central tube available for heat transfer between the supply coolant and the return coolant. In other words, by shrinking the diameter of the central tube to enlarge the annular space around the central tube and thus increase the return coolant flow area, the heat transfer surface area of the central tube is decreased; conversely, by increasing the diameter of the central tube to enlarge the heat transfer surface area of the central tube, the annular space around the central tube, and thus the return coolant flow area, is reduced.

Further, due to poor insulation, many attempts at cryogenic liquid probes have failed in practice due to vapor lock. In such cases, a probe may operate satisfactorily when initially supplied with cryogenic liquid. However, if the operator pauses momentarily in using the probe, some of the cryogenic liquid in the supply or return passageways may vaporize, thereby preventing further cryogenic liquid from flowing through the probe.

### SUMMARY

An insulated cooling probe is disclosed that overcomes the limitations of prior probes. In particular, the coolant supply is provided through an outer annulus between a vacuum insulated sleeve and a return tube. Because the surface area for cooling is now between about 2 and about 20 times larger than in prior probes and flow restriction is significantly reduced in the coolant return, far greater pre-cooling of the supply coolant is achieved.

An insulated cooling probe is provided in which a highly evacuated insulated sleeve is used, in combination with an annular supply passageway surrounding a tubular return passageway, to achieve much higher cooling efficiencies and better controlled cooling at the probe tip when compared with prior designs, as well as to avoid problems of vapor locking when the probe is used with cryogenic liquid flowing in the supply and return passageways.

In one embodiment, an insulated cooling probe is provided having a probe sleeve assembly, a cooling tip, and a heat transfer region. The probe sleeve assembly includes an annular insulating jacket having an inner jacket wall spaced apart by an insulating space from an outer jacket wall, the jacket walls defining at an end of the insulating jacket an evacuation vent and being formed such that a deeper vacuum is achieved within the insulating space than that applied to the vent. The probe sleeve assembly further has a coolant inlet passageway and a coolant exit passageway bounded by a coolant passageway wall surrounded by the insulating jacket, and an orifice across which the coolant expands upon exiting the coolant inlet passageway and entering the cooling tip. The cooling tip extends outwardly from the sleeve assembly at one end of the probe, and includes an expansion chamber cooled by expansion of coolant across an orifice joining the coolant inlet passageway and the coolant exit passageway. The heat transfer region enables pre-cooling of the coolant flowing in the coolant inlet passageway by transferring heat to coolant flowing in the coolant exit passageway.

In one embodiment, the heat transfer region is defined by the coolant passageway wall. In another embodiment, the heat transfer region includes a high heat transfer coefficient surface on the coolant passageway wall bounding the coolant exit passageway. In yet another embodiment, the heat transfer region includes a coil disposed in intimate contact with the coolant passageway wall in the coolant exit passageway. Alternatively, the heat transfer region includes a finned heat exchange device disposed in intimate contact with the coolant passageway wall in the coolant exit passageway. As a further alternative, the heat transfer region includes a metal wool positioned in the coolant exit passageway in intimate contact with the coolant passageway wall. As yet a further alternative, the heat transfer region includes a spiral groove pressed into a coolant passageway wall such that turbulence-creating ridges and valleys are formed on the supply and return sides of the wall.

In another embodiment, an insulated cooling probe is provided having a probe sleeve assembly, a cooling tip, and a heat transfer region. The probe sleeve assembly includes an annular insulating jacket adapted to achieve a deeper vacuum than that applied to evacuate the jacket, a coolant inlet passageway, and a coolant exit passageway separated by a coolant passageway wall surrounded by the insulating jacket. The coolant inlet passageway is an annulus surrounding the coolant exit passageway. A cooling tip extends outwardly from the sleeve assembly at one end of the probe. Coolant expands across an orifice upon exiting the coolant inlet passageway and entering the cooling tip. The cooling tip includes a cooling region into which coolant enters from the coolant inlet passageway and from which coolant exits into the coolant exit passageway, the cooling region being capable of applying targeted cooling to a subject tissue. The heat transfer region is disposed along the coolant passageway wall to enable pre-cooling of the coolant flowing in the coolant inlet passageway by transferring heat to coolant flowing in the coolant exit passageway. One or both of the cooling tip and the coolant passageway wall is moveable with respect to the probe sleeve assembly to control the size and shape of the cooling region on the cooling tip.

In another embodiment, an insulated probe sleeve assembly is provided having an annular insulating jacket including an inner jacket wall spaced apart from an outer jacket wall, and an evacuation vent at an end of the insulating jacket. The inner jacket wall and the outer jacket wall are shaped in the vicinity of the evacuation vent to achieve a deeper vacuum within the insulating space than that applied to the evacuation vent.

In another embodiment, a method is provided for cryogenically cooling a subject tissue via a cooling tip by using an insulated cooling probe. The insulated cooling probe includes an annular insulating jacket adapted to achieve a deeper vacuum than that applied to evacuate the jacket, a coolant passageway wall surrounded by the insulating jacket and forming a tubular passageway inside the coolant passageway wall and an annular passageway formed between the coolant passageway wall and the insulating jacket. The tubular passageway and the annular passageway are joined by the cooling tip. The method includes flowing a higher pressure supply coolant into the annular passageway formed between the coolant passageway wall and the insulating jacket, maintaining a lower back pressure in the tubular passageway inside the coolant passageway wall, expanding the higher pressure supply coolant across an orifice from the annular passageway into the cooling tip, resulting in a lower pressure and colder return coolant in the cooling tip, and flowing the lower pressure and colder return coolant in the tubular passageway such that heat is transferred across the coolant passageway wall from the higher pressure supply coolant to the lower pressure return coolant.

### BRIEF DESCRIPTION OF THE DRAWINGS

The above and other aspects, features and advantages of the cooling probe described herein will be more apparent from the following more particular description thereof, presented in conjunction with the following drawings wherein:

FIG. 1 is a partial cross-sectional view of an embodiment of a vacuum insulated cooling probe.

FIG. 2 is a partial sectional view, in perspective, showing an expansion chamber of the cooling probe of FIG. 1.

FIG. 3 is a partial cross-sectional view showing a sleeve assembly portion of an embodiment of a vacuum insulated cooling probe showing a fluid inlet configuration.

FIG. 4 is a partial cross-sectional view of an embodiment of a vacuum insulated cooling probe showing an integral cooling tip on the sleeve assembly of FIG. 3.

FIG. 5 is a partial cross-sectional view showing an embodiment of a cooling probe showing replaceable trocar tip on the sleeve assembly of FIG. 3.

FIG. 6 is a partial cross-sectional view showing an embodiment of a cooling probe including a metal coil heat exchange region.

FIG. 7 is a partial cross-sectional view showing an embodiment of a cooling probe including a serrated fin heat exchange region.

FIG. 8 is a partial cross-sectional view showing an embodiment of a cooling probe including a metal wool heat exchange region.

FIG. 9 is a cross-sectional view showing an embodiment of a cooling probe for use with liquid coolant.

FIGS. 10A-10E depict various tip configurations that can be used in conjunction with a vacuum insulated probe sleeve assembly, such as shown in FIG. 3, for targeting the application of cooling to subject tissue.

FIG. 11 is a partial cross-sectional view showing an embodiment of a cooling probe including a spiral grooved heat exchange region.

### DETAILED DESCRIPTION

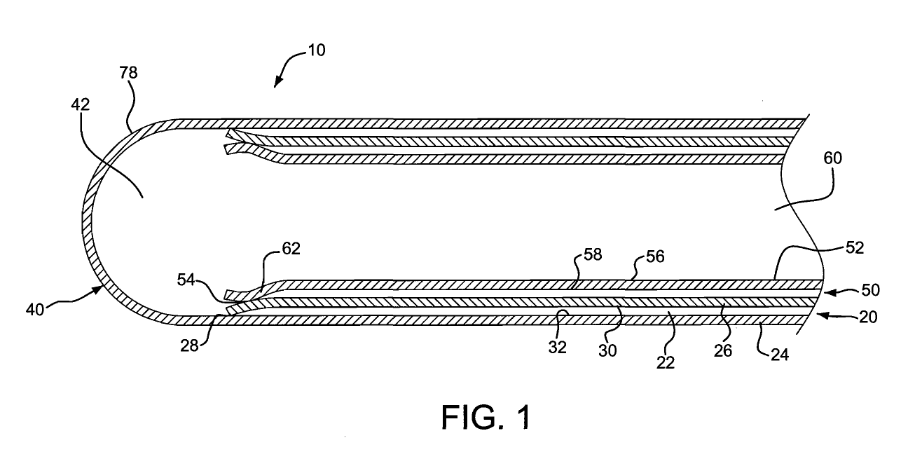

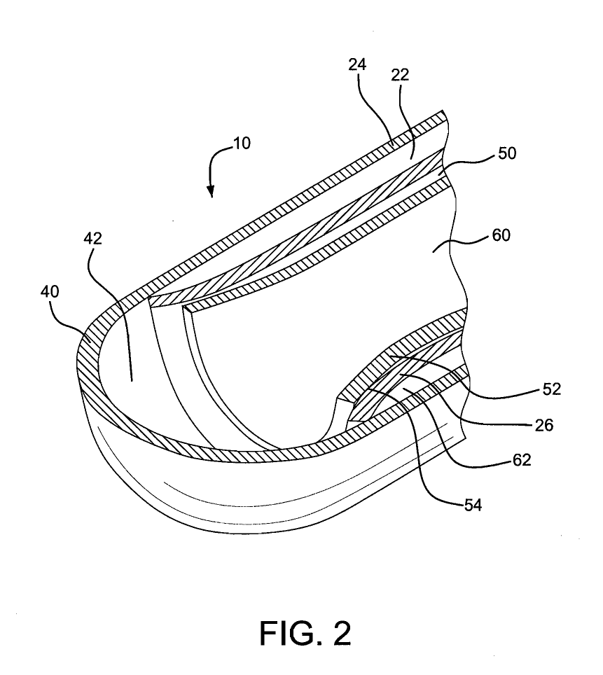

A front end or working end of a vacuum insulated cooling probe 10 is shown in FIGS. 1 and 2. The probe 10 includes a vacuum insulation jacket 20, a probe tip 40, a coolant inlet passageway 50 and a coolant exit passageway 60. A fluid coolant, gas or liquid, is provided to the coolant inlet passageway 50.

The insulation jacket 20 is formed by two substantially concentric tubes, an outer jacket wall 24 and an inner jacket wall 26, which enclose a substantially annular insulating space 22. A vent 28 is disposed between corresponding ends of the outer jacket wall 24 and the inner jacket wall 26 to enable the insulating space 22 to be evacuated by applying a vacuum to the vent 28. The outer jacket wall 24 and the inner jacket wall 26 are configured to work in conjunction with one another to enable a depth of vacuum to be achieved within the insulating space 22 that is greater than the vacuum applied to evacuate the insulating space 22 via the vent 28. In particular, as described below and in commonly owned U.S. Pat. Nos. 7,374,063 and 7,681,299, the relative geometry of the walls 24 and 26 adjacent to the vent 28 has a guiding effect on gas molecules in a free molecular flow regime so that the flux of gas molecules out the vent 28 is greater than the flux of gas molecules into the vent 28. A highly insulated space having a low vacuum created by such geometry can be used in devices of miniature scale or in devices having insulating spaces of extremely narrow width. For example, insulating spaces 22 have been created incorporating this geometry having gaps between the walls 24 and 26 on the order of 0.002 inches or smaller. The insulating space 22 of the insulating jacket 20 is evacuated prior to using the probe 10 for a cooling application.

More specifically, an exemplary gas molecule guiding geometry is depicted in FIG. 1. In gases under relatively modest vacuums, for example at pressures equal to or greater than about 10⁻² torr at about 70° F., molecule-to-molecule collisions dominate such that the number of interactions between the gas molecules themselves is large in comparison to the number of interactions between the gas molecules and the walls of a container for the gas molecules. In this circumstance, Maxwell's gas law accurately describes the molecular kinetic behavior of gas molecules. However, at greater (deeper) levels of vacuum, for example at pressures less than about 10⁻² torr, and particularly at pressures less than about 10⁻⁴ torr at about 70° F., a free molecular flow regime takes over because the scarcity of gas molecules causes the number of interactions between the gas molecules and the walls of the container to be large in comparison with the interactions between the gas molecules themselves. At such low pressures, the geometry of a space to which vacuum is applied becomes a controlling factor in the rate at which gas molecules exit the space via a vent as compared with the rate at which gas molecules enter the space via the vent.

The geometry of the insulation jacket 20 near the vent 28 guides gas molecules within the insulating space 22 toward the vent 28. In particular, the inner jacket wall 26 converges toward the outer jacket wall 24 approaching the vent 28. In addition, while vacuum is being applied to the vent 28, the probe 10 may be heated to accelerate the motion of the gas molecules within the insulating space 22, so as to further bias the flux of gas molecules outward from the vent 28 as compared with inward into the vent 28. For example, the probe 10 may be heated to an elevated temperature and held at that temperature for a period of time during the evacuation process. Longer hold times may be used to further increase the vacuum achievable in the jacket.

The resultant vacuum that is achieved within the insulating space 22 is at a deeper vacuum (i.e., a lower pressure, closer to complete vacuum) than the level of vacuum applied external to the vent 28. This somewhat counterintuitive result is caused by the geometry of the jacket walls 24 and 26 adjacent to the vent 28, which significantly increases the probability that a gas molecule, in the free molecular flow regime occurring at very low pressures, will leave rather than enter the insulating space 22. In effect, the geometry of the jacket walls 24 and 26 functions like a partial check valve to facilitate free passage of gas molecules in one direction (outward from the insulating space 22 via the vent 28) while inhibiting passage in the opposite direction.

Once a desired level of vacuum has been achieved in the insulating space 22, the vent 28 is sealed to maintain the vacuum. In one embodiment, the vent 28 is sealable by a brazing material that melts and flows into the vent 28 when heated to a brazing temperature, so that the ends of the inner jacket wall 26 and outer jacket wall 24 are brazed together and the insulating space 22 is sealed off. The use of brazing to seal the evacuation vent of a vacuum-sealed structure is generally known in the art. To seal the vent 28, a brazing material (not shown) is positioned between the inner jacket wall 26 and the outer jacket wall 24 adjacent to their ends in such a manner that during the evacuation process (i.e., prior to the brazing process) the vent 28 is not blocked by the brazing material. Toward the end of the evacuation process, as the desired level of vacuum is being achieved in the insulating space 22, sufficient heat is applied to the probe 10 to melt the brazing material such that it flows by capillary action into the vent 28. The flowing brazing material seals the vent 28 and blocks the evacuation path from the insulating space 22. Flowing of the brazing material is facilitated by any preheating that occurs by heating of the probe 10 during the evacuation phase in order to enhance the ultimate level of vacuum achieved in the insulating space 22. Alternatively, other processes can be used for sealing the vent 28, including but not limited to a metallurgical process or a chemical process.

By being able to achieve a deep vacuum due to the geometry of the insulation jacket 20 and without need for a getter material, the insulating space 22 (a function of the radial distance between the outer jacket wall 24 and the inner jacket wall 26) can be kept very small, for example on the order of a few thousands of an inch, which in turn allows for miniature probes and other devices using such an insulation jacket 20.

To enhance the insulating properties of the sealed evacuated insulating space 22, an optical coating 30 having low-emissivity properties may be applied to an outer surface of the inner jacket wall 26 and/or to an inner surface of the outer jacket wall 24 to limit radiative heat transfer across the insulating space 22. Any low emissivity surfaces known in the art can be used.

The probe tip 40 is adjoined to the outer jacket wall 24 of the insulation jacket 20, and encloses an generally hemispherical expansion chamber 42, although equivalent expansion chambers 42 of various geometries can be provided. A coolant passageway wall 52 is disposed substantially concentrically with the insulation jacket 20, radially inward from the inner jacket wall 26. The coolant inlet passageway 50 is formed between the coolant passageway wall 52 and the inner jacket wall 26, and the coolant outlet passageway 60 is bounded by the coolant passageway wall 52. For a given inside diameter of the inner jacket wall 26, the thickness and diameter of the coolant passageway wall 52 are sized with consideration of the ratio of the coolant outlet passageway 60 cross-sectional area to the coolant inlet passageway 50 cross-sectional area and the heat transfer surface area between the passageways 50, 60. As a result of the presently disclosed configuration, both parameters can be optimized at the same time, in contrast to prior designs in which one parameter was improved at the expense of the other. In particular, in an embodiment of the cooling probe described herein, increasing the diameter of the coolant passageway wall 52, assuming the thickness of the wall 52 is held constant, increases both the coolant return-to-supply flow area ratio and the heat transfer surface area, since the central tube inner diameter increases while the annular gap between the coolant passageway wall 52 and the inner jacket wall 62 decreases. In contrast, in prior designs, increasing the diameter of the coolant passageway wall would increase the heat transfer area at the expense of the coolant return-to-supply flow area (which is the inverse of the coolant return-to-supply flow area described herein). In one embodiment, the coolant return-to-supply flow area ratio is equal to or greater than 1. In other embodiments, the coolant return-to-supply flow area ratio can range between about 2.7 and about 36, typically between about 2.7 and about 15, and most typically between about 2.7 and about 10.

A diffuser or orifice 54, or a plurality of diffusers or orifices 54, located at or near an end portion 62 of the coolant passageway wall 52 connects the coolant inlet passageway 50 and the coolant outlet passageway 60. Coolant fluid, in the form of a liquid or gas, is introduced into the coolant inlet passageway 50 and is expanded through the orifice to cool the expansion chamber 42. Because the coolant can be a liquid or gas, the terms inlet fluid and inlet gas are used interchangeably herein, it being understood that the inlet fluid or inlet gas could also be an inlet liquid. As is known in the art, a fluid experiences a significant temperature drop upon adiabatic expansion across an orifice from a higher pressure to a lower pressure. The expanded fluid (typically a gas but sometimes a liquid) flows out of the expansion chamber 42 through the coolant exit passageway 60. As shown in FIG. 1, the diffuser 54 can be a small annular gap between the coolant passageway wall 52 and the insulating jacket 20. Alternatively, one or more orifices 54 can be provided in the coolant passageway wall 52 between the coolant inlet passageway 50 and the coolant outlet passageway 60.

The adiabatic expansion cooling of the fluid across the orifice 54 causes the probe tip 40 to be cooled. The probe tip 40 can thus be used to provide targeted cooling to a subject surface, such as for medical treatment or other applications. The expanded low-temperature, low-pressure fluid (gas or liquid) is exhausted through the coolant outlet passageway 60. As the low-temperature fluid passes along an inner surface 56 of the coolant passageway wall 52, the wall 52 is chilled. In turn, the fluid passing along an outer surface 58 of the coolant passageway wall 52 flowing toward the orifice 54 is chilled by the wall 52. In other words, heat from the high-pressure inlet fluid flowing in the coolant inlet passageway 50 is conducted through the wall 52 and into the lower-pressure outlet or exhaust fluid flowing in the coolant outlet passageway 60, such that the wall 52 acts as a counterflow heat exchanger. Therefore, by cooling the inlet fluid using the outlet fluid, the temperature achieved in the expansion chamber 42 can be reduced. In attempt to achieve cooling similar to that achieved herein, prior designs have needed to utilize separate and more complex heat exchangers located in cooling tip or in a probe handle distal from the tip. Additionally, the vacuum insulating jacket 20 inhibits heat gain by the inlet fluid from the exterior of the probe. This reduction in heat gain may be enhanced by applying a coating of emissive radiation shielding material on the outer surface of outer jacket wall 24.

The ability to improve both the coolant return-to-supply flow area ratio and the heat exchange surface area at the same time provides significant benefits with regard to improving the cooling efficiency of the probe. First, a larger return-to-supply flow area ratio decrease the back-pressure on the expanded coolant fluid flowing in the coolant outlet passageway 60, thereby allowing a greater pressure drop across the orifice 54 which results in greater cooling effect due to the expansion. Second, a larger heat transfer surface area improves the quenching of the supply coolant flowing in the coolant inlet passageway 50. Thus, the lower pressure expanded fluid is not only colder, but is enabled to transfer more cooling back to the higher pressure supply fluid, creating a positive feedback loop that continues to make the expanded fluid even colder. Additionally, in the probe design disclosed herein, beneficial results can actually be obtained by making the probe longer, because increased length results in only a minimal increase in pressure drop in the coolant outlet passageway 60 but a large increase (proportional to length) in the heat transfer surface area. In contrast, in prior prove designs having a small coolant return-to-supply flow area ratio, increasing the length of the probe incurred significant penalties in increased pressure drop (and back pressure) in the coolant outlet passageway, which outweighed any benefits of increased heat transfer surface area.

With reference to FIG. 2, the end portion 62 of the coolant passageway wall 52 which forms the orifice 54 may be adapted to flex in response to pressure applied by the inlet fluid. In this manner, the size of the opening defined by the orifice 54 between inner jacket wall 26 and the coolant passageway wall 52 may be varied in response to variation in the fluid pressure within inlet passageway 50. A higher pressure fluid is introduced into the coolant inlet passageway 50, expands through the orifice 54 into the expansion chamber 42 to cool the probe tip 40, and is exhausted as a lower pressure fluid through the coolant exit passageway 60.

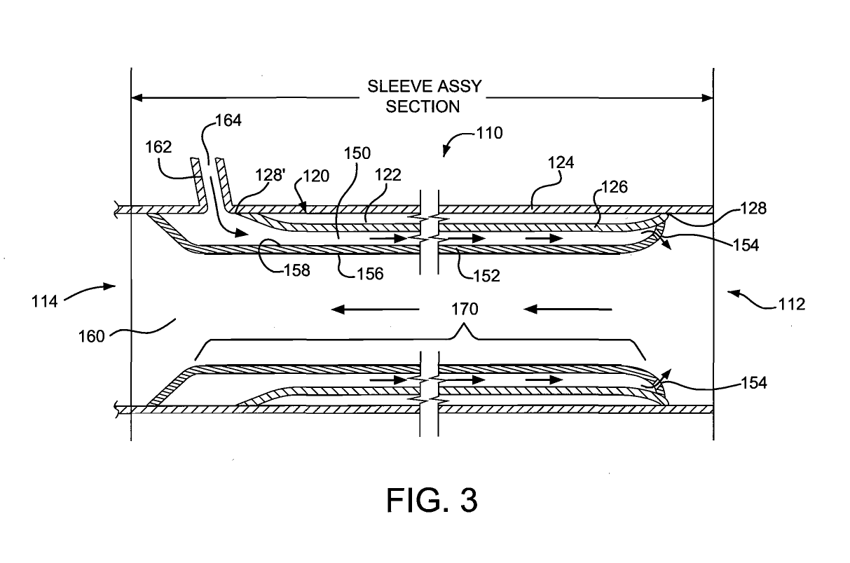

FIG. 3 shows a cooling sleeve assembly 110 for a cooling probe that can be used with any of a variety of probe tips. The sleeve assembly 110 has a front end 112 at which a probe tip would be attached or affixed, and a rear end 114 extending in a direction opposite the probe tip. The sleeve assembly 110 includes an insulating jacket 120 having an outer jacket wall 124 and an inner jacket wall 126 that are disposed substantially concentrically with respect to each other to form an annular insulation space 122 therebetween. The insulation space 122 can be evacuated, as discussed above, through a vent 128 or a vent 128′ to achieve a vacuum deeper than that applied to one or both of the vents 128 and 128′, due to the geometric configuration of the inner wall 124 and outer wall 126 adjacent to one or both of the vents 128 and 128′. The sleeve assembly 110 can be of any length, as indicated by the broken middle section, including as long as many feet. The sleeve assembly 110 can be rigid or flexible to provide for a desired positioning of a probe tip during use of the probe. Using an insulating jacket 120 as described herein, the length-to-diameter ratio of a probe is virtually unlimited, and may be on the order of one hundred, several hundred, or even a few thousand. For example, probes have been built having an outside diameter of less than 1.5 millimeter (about 0.060 inches) and a sleeve assembly 110 more than nine feet long.

The sleeve assembly 110 further includes a coolant passageway wall 152 disposed within, and substantially concentric with, the insulation jacket 120. An annular coolant inlet passageway 150 is formed between the coolant passageway wall 152 and the inner jacket wall 126. High pressure inlet fluid (gas or liquid) is supplied to the coolant inlet passageway 152 by an inlet conduit 162 extending through the outer wall 124 rearwardly from a rear end of the insulating jacket 120, the inlet conduit 162 forming an inlet 164. The inlet conduit 162 can be connected to any source of coolant fluid. A generally cylindrical coolant outlet passageway 160 is formed interior to the coolant passageway wall 152 for allowing low pressure fluid to exit the sleeve assembly 110 after adiabatic expansion. One or more diffusers or orifices 154 are disposed toward a front end of the coolant passageway wall 152 through which the high pressure fluid from the coolant inlet passageway 152 is adiabatically expanded into an expansion chamber in the probe tip, creating a chilling effect. The number and size of the orifices 154 can be selected based on the desired chilling effect, which depends on several factors, including but not limited to the pressure of the inlet fluid, the back-pressure of the outlet fluid, the flow rate of the inlet fluid, the state of the inlet fluid, and the initial temperature of the inlet fluid. In particular, the available coolant supply pressure combined with the coolant outlet back pressure bounds the maximum pressure expansion ratio, an advantage in the present design which improves the coolant return-to-supply flow area ratio and thus decreases the coolant outlet back pressure. A benefit of the probe sleeve 110 is that more coolant can be effectively used than in prior designs to achieve more cooling as a result of the lower coolant outlet back pressure.

It is understood that a cooler inlet fluid generally will result in fluid of a lower temperature immediately after the fluid has been adiabatically expanded across the orifices 154. In turn, the temperature of the adiabatically expanded fluid controls the temperature of a probe tip that is attached to the sleeve assembly 110. However, it is also understood that in order to maintain the expansion cooling, a continual flow of fluid must be expanded across the orifices 154. Because the amount of heat that can be transferred from a probe tip to the chilled fluid is limited by several factors, most importantly the overall heat transfer rate at which heat can be transferred from a subject with which the probe tip is in contact (via conduction and convention), through the probe tip itself (via conduction), and from the probe tip to the chilled fluid in the probe tip expansion chamber (via convection), the temperature of the low-pressure outlet fluid exhausted through the coolant outlet passageway 160 will still be low relative to the temperature of the inlet fluid. Accordingly, to the extent that adiabatic expansion cooling can be recovered from the outlet fluid by quenching the inlet fluid, based in part on the size and characteristics of a heat exchange region 170 described below, the cooling efficiency of the sleeve assembly 110 can be improved to result in a cooler, and thus a more effective, probe tip.

A heat exchange area 170 is defined along a portion of the cooling passageway wall 152 separating the high-pressure relatively warmer inlet fluid flowing toward the orifice 154 and the probe tip from the low-pressure relatively cooler outlet fluid flowing away from the orifice 154 and the probe tip. In the heat exchange area 170, heat is transferred by convention from the inlet fluid in the inlet passageway 150 to an outer surface 158 of the coolant passageway wall 152, by conduction through the wall 152, and by convention from an inner surface 156 of the wall 152 to the outlet fluid in the outlet passageway 160, thereby recovering some of the chilling achieved in the adiabatic expansion of the inlet fluid across the orifice 154 by pre-chilling or quenching the inlet fluid. However, the amount of cooling that can be recovered may be limited, particularly on the outlet side (i.e., along the inner surface 156 of the wall 152), particularly if the outlet fluid is gaseous, by the low convective heat transfer coefficient of the low-pressure (low-density) outlet fluid.

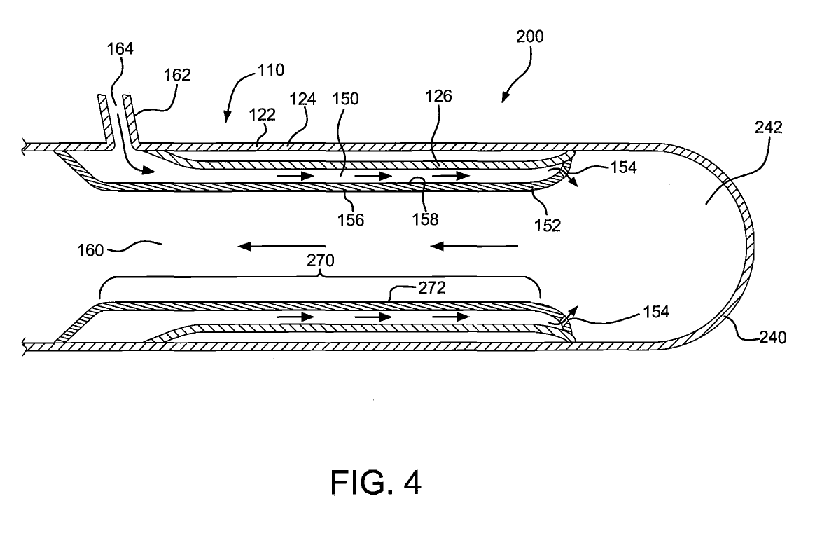

FIG. 4 shows an embodiment of a cooling probe 200 including a substantially hemispherical probe tip 240 integrally mounted to the sleeve assembly 110. The sleeve assembly 110 has a heat transfer region 270. A cooling chamber 242 is formed within the probe tip 240 for receiving the chilled adiabatically expanded cooling fluid and for providing sufficient retention time so that heat can be transferred through the probe tip 240 into the cooling fluid before the fluid is exhausted through the coolant outlet passageway 160. As discussed above, in using the sleeve assembly 110 including the expansion orifices 154, the temperature to which the cooling chamber 242 can be cooled, for any given coolant, and total orifice area, is dependent in part on the temperature of the inlet fluid provided by the coolant inlet passageway 150 to the orifice 154. In one embodiment, the heat transfer region 270 is treated with a high heat transfer surface 272, such as a surface having high conductivity and a surface roughness to promote turbulent flow (with a commensurately higher heat transfer coefficient than laminar flow). It is noted that although the sleeve assembly 110 is depicted as being relatively short in length, the sleeve assembly 110, including the insulating jacket 120 and the coolant inlet and outlet passageways 150 and 160, respectively, can extend for a long distance, such as several feet, from a source of coolant to the probe tip 240. For example, a probe have been constructed having a sleeve assembly 110 over nine feet long, in which the cooling effect was not significantly diminished due to the extremely effective insulating properties of the insulating jacket 120 capable of achieving a very low vacuum in the insulation space 122, as discussed above.

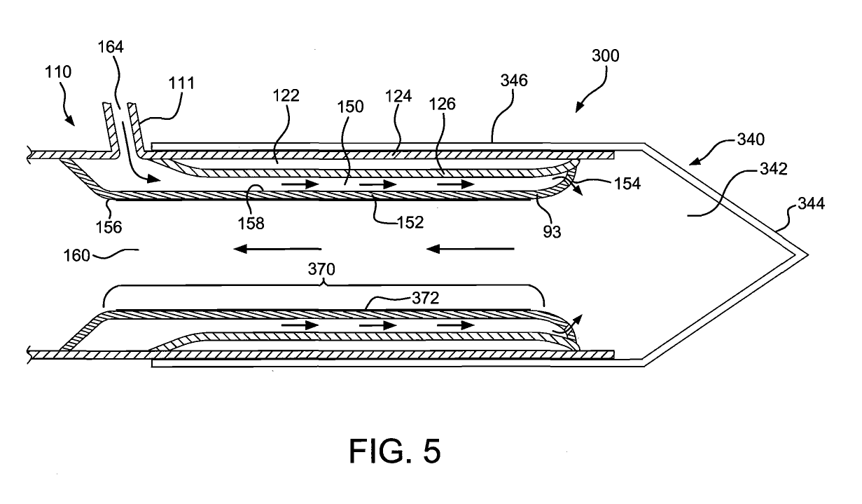

FIG. 5 shows an embodiment of a cooling probe 300 including a removably mounted trocar tip forming the probe tip 340. A cooling chamber 342 is formed within the probe tip 340. The probe tip 340 includes a contact tip 344 and a substantially cylindrical shell 346 extending rearwardly from the contact tip 344, the shell 346 being slidably received over a front portion of the sleeve assembly 110. A seal can be formed between the shell 346 and the sleeve assembly 110 by o-rings, by brazing, or by other methods known in the art. In some instances, brazing of the tip 340 to the shell 346 may be preferable because it anneals the metal of the tip to make it more resistant to ultra-low temperatures and overcomes any sealing difficulties that may result from different coefficients of thermal expansion in the sleeve assembly 110 and the tip 340. The sleeve assembly 110 includes a heat transfer region 370. In one embodiment, a high heat transfer surface 372 can be provided on the inner surface 156 of the coolant passageway wall 152 to enhance heat exchange between the inlet fluid and the outlet fluid.

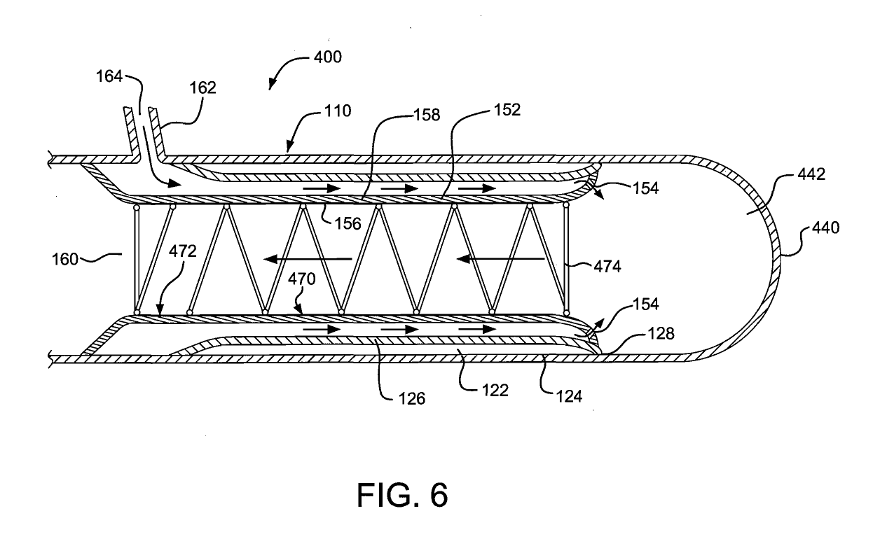

In order to achieve enhanced cooling in a probe tip, the inlet fluid temperature can be reduced, which will in turn reduce the temperature of the fluid that is adiabatically expanded across the orifice 54. In particular, because the inlet fluid is typically either a liquid or a high pressure (relatively higher density) gas, and the outlet fluid is typically a low pressure (lower density) liquid or gas, the heat transfer coefficient between the inlet fluid and the outer surface 58 of the coolant passageway wall 52 may be significantly greater than the heat transfer coefficient between the outlet fluid and the inner surface 56 of the coolant passageway wall 52. In other words, the overall heat transfer coefficient from the outlet fluid to the inlet fluid is limited primarily by the heat transfer coefficient between the outlet fluid and the passageway wall 52. Therefore, it is desirable to provide configurations to enhance the heat transfer coefficient, or the rate of heat transfer, between the outlet fluid and the inner surface 56 of the coolant passageway wall 52 in order to achieve an enhanced overall precooling of the inlet fluid before it is expanded across the orifice 54. Exemplary configurations of sleeves are provided in the embodiments of FIGS. 6, 7, and 8 to improve cooling recovery to the inlet fluid.

FIG. 6 shows a probe 400 having a coil 472 disposed within a heat transfer region 470 to enhance the heat transfer between the inlet fluid flowing in the coolant inlet passageway 150 and outlet fluid flowing in the coolant outlet passageway 160. The coil 472 is disposed in intimate heat transfer contact with the inner surface 156 of the coolant passageway wall 152 so as to provide rapid conduction heat transfer between the coil 472 and the wall 152. As shown, the coil 472 is generally spirally wound against the surface 156, it being understood that separate segments of the coil 472 could alternatively be formed generally perpendicularly to the direction of outlet fluid flow. In one embodiment, the coil 472 is affixed to the wall 152 by brazing. As the outlet fluid flows through the coolant outlet passageway 160, the boundary layer of fluid along the inner surface 156 is repeatedly broken up by successive segments of the coil 472, causing turbulence and restarting of the boundary layer, thereby significantly increasing the heat transfer coefficient between the outlet fluid and the wall 152. Because, as discussed above, the overall heat transfer from the outlet fluid to the inlet fluid is likely to be most limited by the convective heat transfer coefficient between the low pressure outlet fluid and the wall 152, particularly if the outlet fluid is a gas (as compared with conduction through the wall 152, which has a high heat transfer coefficient, and convection between the wall 152 and the inlet fluid, which will have a higher heat transfer coefficient due to the higher pressure of the inlet fluid, particularly if the inlet fluid is a liquid), the overall heat transfer coefficient is increased.

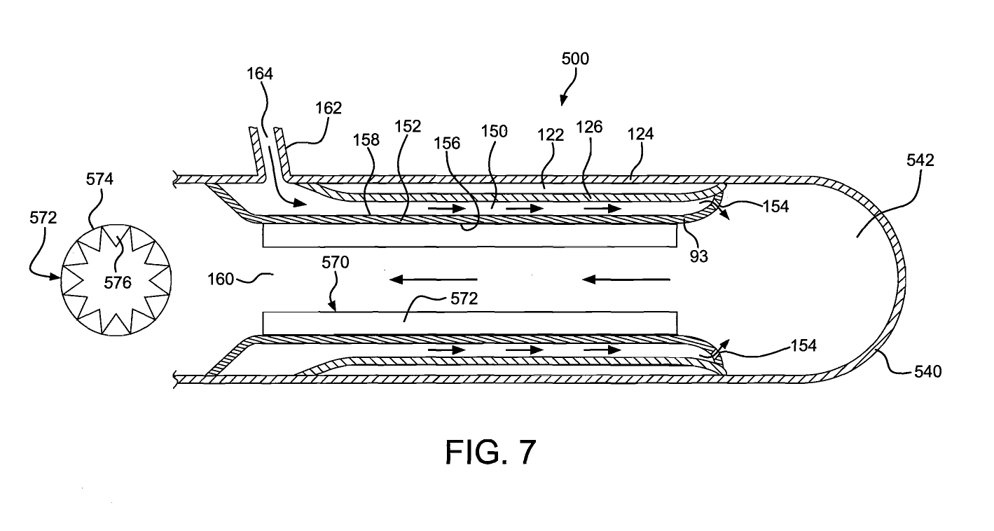

FIG. 7 shows a probe 500 having a serrated finned heat transfer device 472 within a heat transfer region 570 to enhance the heat transfer between the inlet fluid flowing in the coolant inlet passageway 150 and the outlet fluid flowing in the coolant outlet passageway 160. The heat transfer device 572 includes an outer surface 574 in intimate contact with the inner surface 156 of the coolant passageway wall 152 and a plurality of fins 576 extending radially inward into the outlet passageway, the fins 576 being disposed to run generally parallel to the outlet fluid flow. The fins 576 provide a greater heat transfer surface area through which the outlet fluid can transfer heat to the wall 152, and also create more turbulent flow (with its commensurate higher heat transfer coefficient) than would be achieve without the heat transfer device 572. As shown, the fins 576 each have a generally triangular cross-section with a broader base tapering to a narrower tip, as is common on heat transfer fins.

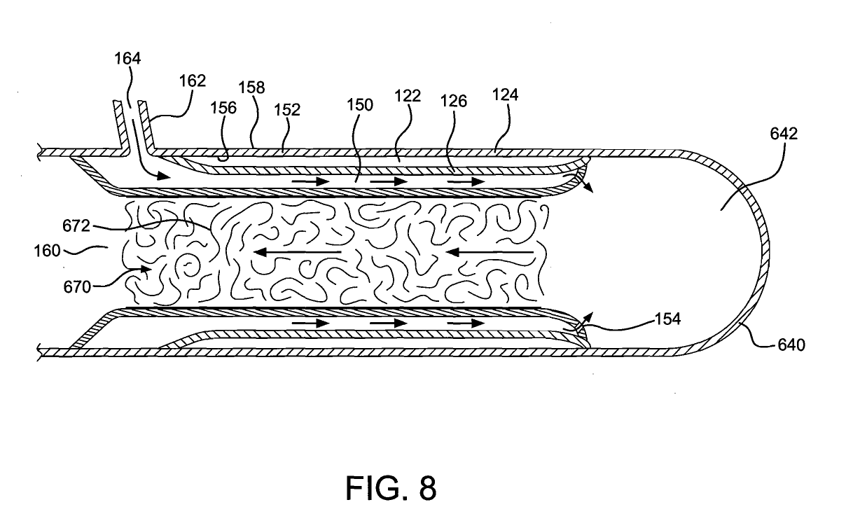

FIG. 8 shows a probe 600 having a metal wool 672 within a heat transfer region 670 to enhance the heat transfer between the inlet fluid flowing in the coolant inlet passageway 150 and the outlet fluid flowing in the coolant outlet passageway 160. The metal wool 672 is positioned in the coolant passageway so as to be in intimate contact with the surface 156 of the coolant passageway wall 152. The metal wool 672 creates a tortuous path for the outlet fluid resulting in a high heat transfer coefficient. Metal wools 672 of various metals can be used, including cooper, aluminum, and stainless steel.

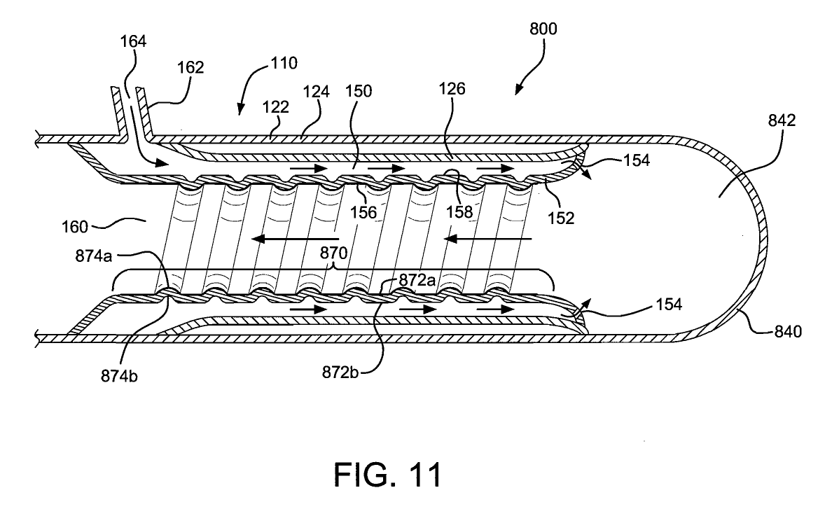

FIG. 11 shows a probe 800 having a spiral grooved heat transfer region 870 to enhance the heat transfer between the inlet fluid flowing in the coolant inlet passageway 150 and the outlet fluid flowing in the coolant outlet passageway 160. The spiral grooving can be achieved by pressing a tool against the outer surface 158 of the tubular coolant passageway wall 152 as the tube is rotated. The spiral grooved region 870 creates peaks 874a and troughs 872a in the coolant return passageway 150 (which correspond to troughs 874b and peaks 872b in the coolant return passageway 160), which creates enhanced turbulence and more surface area, and thus increased heat transfer, on both sides of the coolant passageway wall 152.

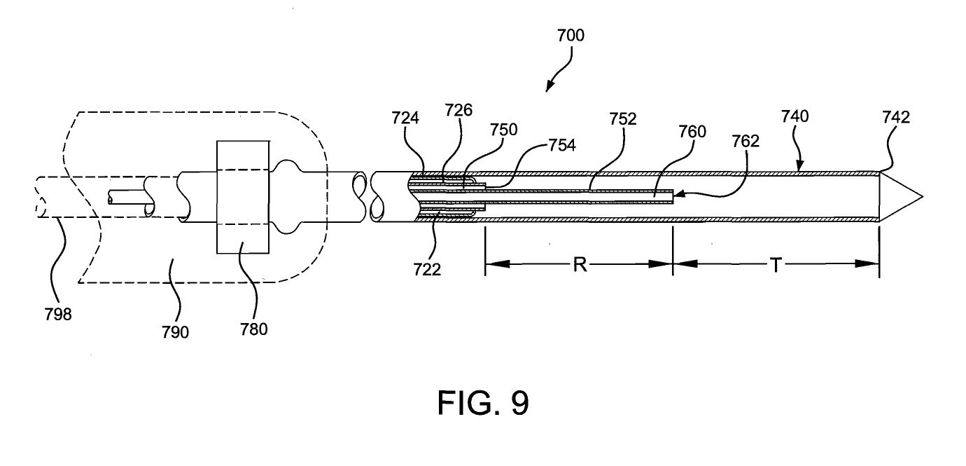

FIG. 9 depicts an embodiment of a probe 700 particularly designed for use with liquid coolant undergoing adiabatic expansion. Similarly to the probes discussed above, the probe 700 includes a concentric tube structure that forms a vacuum insulated space 722 enclosing a coolant supply passageway 750 and a coolant return passageway 760, and provides cooling to a tip 740. The vacuum insulated space 722 between tubes 724 and 726 is evacuated as described above. Liquid coolant is supplied in the annular passageway 750 between tube 726 and tube 752, and after exiting the supply passageway 750 and expanding across the opening 754 into the cooling the tip 740, the liquid coolant returns through the passageway 760 inside the tube 752. The tip 740 can be in the form of a replaceable sleeve, or can be integral with the main portion of the probe 700.

Because of the highly effective insulated space 722, vapor locking of the probe 700 can be avoided, even if use of the probe 700 is suspended for a period of time. Without this highly effective insulation, liquid coolant in the supply passageway 750 could at least partially evaporating, causing “vapor lock” in which the flow of liquid coolant is blocked by a vapor bubble. This phenomenon, common in prior attempts at cryogenic liquid probes, typically occurs when the probe is used for a period of time and then use of the probe is suspended—even for a few seconds, e.g., to reposition the probe to be in contact with a different target surface—before attempting to restart liquid flow. In an embodiment of the present probe 700, liquid flow has been suspended for a few seconds, and even for up to about 2 minutes, and then restarted without vapor locking of the coolant flow passageways 750 and 760.

As shown, the tube 752 extends beyond the opening 754 in the supply passageway 750 by a return tube distance R, and a return tube-to-tip distance T is established between a tip end 762 of the tube 752 and a tip end 742 of the probe tip 740. The distances R and T can be adjusted independently or in combination. By adjusting the one or both of the distances R and T, the size of a cooling region on the cooling tip 740 can be controlled. In practice, the cooling region on the cooling tip 740 is often identified by formation of an ice ball during use of the probe. In an example, when the distance T is adjusted to be relatively short, a short concentrated cooling region is created near the tip end 742 of the cooling tip 740. In another example, when the distance R is adjusted to be relatively long, an extended cooling region is created along the length of the cooling tip 740 generally corresponding to the position of the distance R.

A handle 790 is provided for grasping and manipulating the probe 700, and a supply/return tube 798 extends from the handle 798 to a console (not shown) which supplies the cryogenic fluid to the probe and rechills the slightly warmed, lower pressure cryogenic fluid returning from the probe 700. In one variation, the vacuum insulated structure extends only from the handle 790 to near the tip 740. In another variation, the vacuum insulated structure extends all the way from near the tip 740, through the handle 790, and through the supply/return tube 798 to the console. A vacuum insulated structure as disclosed herein has been made and operated at lengths exceeding 9 feet.

In the embodiment shown in FIG. 9, mounted on the probe 700 at or near the handle 790, is a piezoelectric ceramic transducer 780. Such a transducer can be provided on any of the probes disclosed herein, and is not limited to the embodiment shown in FIG. 9. The transducer 780 is used to provide vibration, as desired, to the probe tip 740 to assist in penetration of a tissue to be treated. The transducer 780 can be configured to provide unidirectional vibration, either along the longitudinal axis or perpendicular to the longitudinal axis of the probe tip 740. Alternatively, the transducer 780 can be configured to provide bidirectional or tri-directional vibration along two or three directions, respectively.

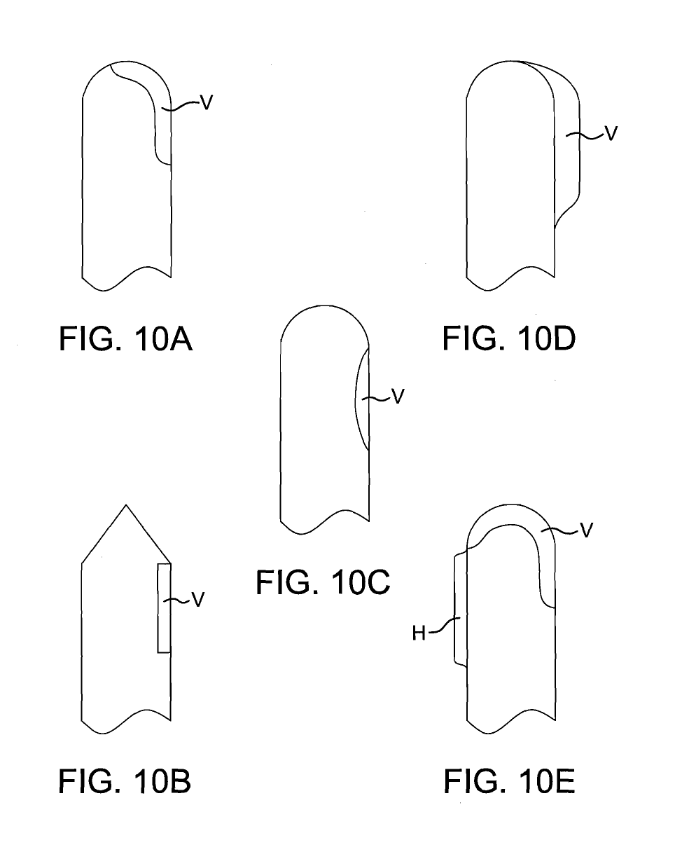

FIGS. 10A through 10E depict several different variations of a probe tip that can be used in conjunction with the probe sleeve assembly 110 depicted generally in FIG. 4. In each variation, a vacuum insulated chamber V is provided having a specific shape and location to enable targeting cooling of some subject tissue while protecting other tissue (i.e., the tissue in contact with the vacuum insulated chamber V) from damage from cooling. The vacuum insulated chambers V can be formed using the same method disclosed above to have a vacuum level deeper than the vacuum applied to vent used to evacuate the chamber.

FIG. 10A shows a vacuum insulated chamber V extending along a portion of the side wall and a portion of the end wall of a rounded probe tip. FIG. 10B shows a vacuum insulated chamber V along a portion of the sidewall of a pointed trocar tip. FIG. 10C shows a vacuum insulated chamber V along a portion of the side wall of a probe tip. FIG. 10D shows a vacuum insulated chamber V positioned outward from a portion of the sidewall of a probe tip. FIG. 11E shows a vacuum insulated chamber V extending along a portion of the sidewall and end wall of a probe tip, in combination with a thermally conductive plate (i.e., and enhanced heat transfer surface) along another portion of the probe tip. It can be appreciated by one of skill in the art that infinite other variations of vacuum insulated chambers V, alone or in combination with enhanced heat transfer surfaces, can be envisioned and fabricated to provide targeted cooling in specific circumstances, according to the therapeutic needs of the subject.

The foregoing describes the cooling probe in terms of embodiments foreseen by the inventors for which an enabling description was available, notwithstanding that insubstantial modifications of the probe, not presently foreseen, may nonetheless represent equivalents thereto.

1. An insulated cooling probe comprising:
a probe sleeve assembly having an annular insulating jacket including an inner jacket wall spaced apart by an insulating space from an outer jacket wall, the jacket walls defining at an end of the insulating jacket an evacuation vent and being formed such that a deeper vacuum is achieved within the insulating space than that applied to the vent, the probe sleeve assembly further including a coolant inlet passageway and a coolant exit passageway separated by a coolant passageway wall surrounded by the insulating jacket;
a cooling tip extending outwardly from the sleeve assembly at one end of the probe, the cooling tip including a cooling region into which higher pressure coolant enters from the coolant inlet passageway and from which lower pressure coolant exits into the coolant exit passageway, the cooling region being capable of applying targeted cooling to a subject tissue;
an orifice across which the coolant expands upon exiting the coolant inlet passageway and entering the cooling tip; and
a heat transfer region that enables pre-cooling of the coolant flowing in the coolant inlet passageway by transferring heat to coolant flowing in the coolant exit passageway.

2. The insulated cooling probe of claim 1, wherein gaseous coolant is supplied to the coolant inlet passageway.

3. The insulated cooling probe of claim 1, wherein liquid coolant is supplied to the coolant inlet passageway.

4. The insulated cooling probe of claim 1, wherein the liquid coolant returns in the coolant exit passageway.

5. The insulated cooling probe of claim 1, wherein the heat transfer region is defined by the coolant passageway wall.

6. The insulated cooling probe of claim 1, wherein the heat transfer region includes a high heat transfer coefficient surface on the coolant passageway wall bounding the coolant exit passageway.

7. The insulated cooling probe of claim 1, wherein the heat transfer region includes a coil disposed in intimate contact with the coolant passageway wall in the coolant exit passageway.

8. The insulated cooling probe of claim 1, wherein the heat transfer region includes a finned heat exchange device disposed in intimate contact with the coolant passageway wall in the coolant exit passageway.

9. The insulated cooling probe of claim 1, wherein the heat transfer region includes a metal wool positioned in the coolant exit passageway in intimate contact with the coolant passageway wall.

10. The insulated cooling probe of claim 1, wherein the heat transfer region includes a spiral grooved wall.

11. The insulated cooling probe of claim 1, wherein the cooling tip is moveable with respect to the orifice to increase or decrease the size and shape of the cooling region on the cooling tip.

12. The insulated cooling probe of claim 1, wherein the coolant inlet passageway is an annulus surrounding the coolant exit passageway.

13. The insulated cooling probe of claim 1, wherein the coolant inlet and coolant outlet passageways are sized to have a coolant return-to-supply flow area ratio of at least 1.

14. The insulating cooling probe of claim 1, wherein the coolant passageway wall extends beyond the coolant inlet passageway into the cooling tip by a return tube length that can be adjusted with respect to the orifice to control the size and shape of the cooling region on the cooling tip.

15. The insulating probe of claim 1, the cooling tip further comprising a vacuum insulated chamber insulating a portion of the surface area of the cooling tip to customize the size and shape of the cooling region on the cooling tip, the cooling region occupying at least a portion of the remaining surface of the cooling tip not insulated by the vacuum insulated section.

16. The insulating probe of claim 15, the cooling tip further comprising a thermally'conducting surface disposed on the cooling region of the cooling tip.

17. The insulating probe of claim 1, wherein the probe has a length and an outer diameter, the length being more than fifty times the diameter.

18. An insulated cooling probe comprising:
a probe sleeve assembly having an annular insulating jacket adapted to achieve a deeper vacuum than that applied to evacuate the jacket, the probe sleeve assembly further including a coolant inlet passageway and a coolant exit passageway separated by a coolant passageway wall disposed within the insulating jacket, the coolant inlet passageway being an annulus surrounding the coolant exit passageway;
a cooling tip extending outwardly from the sleeve assembly at one end of the probe, the cooling tip including a cooling region into which higher pressure coolant enters from the coolant inlet passageway and from which lower pressure coolant exits into the coolant exit passageway, the cooling region being capable of applying targeted cooling to a subject tissue;
an orifice across which the coolant expands upon exiting the coolant inlet passageway and entering the cooling tip; and
a heat transfer region along the coolant passageway wall that enables pre-cooling of the coolant flowing in the coolant inlet passageway by transferring heat to coolant flowing in the coolant exit passageway;
wherein one or both of the cooling tip and the coolant passageway wall is moveable with respect to the probe sleeve assembly to control the size and shape of the cooling region on the cooling tip.

19. An insulated probe sleeve assembly comprising:
an annular insulating jacket including an inner jacket wall spaced apart from an outer jacket wall; and
an evacuation vent at the end of the insulating jacket;
wherein the inner jacket wall and the outer jacket wall are shaped in the vicinity of the evacuation vent to achieve a deeper vacuum within the insulating space than that applied to the evacuation vent.

20. The insulated probe sleeve assembly of claim 19, wherein the depth of the vacuum within the insulating space is increased by heating the probe sleeve assembly during evacuation.

21. A method for providing cryogenic cooling to a subject tissue via a cooling tip by using an insulated cooling probe including an annular insulating jacket adapted to achieve a deeper vacuum than that applied to evacuate the jacket, a coolant passageway wall surrounded by the insulating jacket and forming a tubular passageway inside the coolant passageway wall and an annular passageway formed between the coolant passageway wall and the insulating jacket, the tubular passageway and the annular passageway being joined by the cooling tip, the method comprising:
flowing a higher pressure supply coolant into the annular passageway formed between the coolant passageway wall and the insulating jacket;
maintaining a lower back pressure in the tubular passageway inside the coolant passageway wall;
expanding the higher pressure supply coolant across an orifice from the annular passageway into the cooling tip, resulting in a lower pressure and colder return coolant in the cooling tip; and
flowing the lower pressure and colder return coolant in the tubular passageway such that heat is transferred across the coolant passageway wall from the higher pressure supply coolant to the lower pressure return coolant.
